(19) United States
(12) Patent Application Publication
(10) Pub. No.: US 2025/0387165 A1
(43) Pub. Date: Dec. 25, 2025

(54) DETERMINATION METHOD, APPARATUS, ELECTRONIC DEVICE, AND STORAGE MEDIUM FOR POSTOPERATIVE PORTAL VEIN PRESSURE

(71) Applicants: ZHONGDA HOSPITAL SOUTHEAST UNIVERSITY, Nanjing (CN); University of Shanghai for Science and Technology, Shanghai (CN)

(72) Inventors: Xiaolong QI, Nanjing (CN); Tianqi WANG, Shanghai (CN); Yi XIANG, Nanjing (CN); Yang SHEN, Shanghai (CN); Chuan LIU, Nanjing (CN); Jiaqi GU, Nanjing (CN); Ling YANG, Nanjing (CN)

(73) Assignees: ZHONGDA HOSPITAL SOUTHEAST UNIVERSITY, Nanjing (CN); University of Shanghai for Science and Technology, Shanghai (CN)

(21) Appl. No.: 18/945,579

(22) Filed: Nov. 13, 2024

(30) Foreign Application Priority Data
Jun. 19, 2024 (CN) 202410793487.7

(51) Int. Cl. A61B 34/10; G06T 17/00

(52) U.S. Cl. CPC A61B 34/10; G06T 17/00; A61B 2034/104; A61B 2034/105; A61B 2034/108

(57) ABSTRACT

A determination method, an apparatus, an electronic device, and a storage medium for a postoperative portal vein pressure are provided, wherein the determination includes obtaining preoperative imaging data and measurement data of a patient; and inputting the preoperative imaging data and the measurement data of the patient into a portal vein geometric multi-scale model to obtain a postoperative portal vein pressure of the patient output by the portal vein geometric multi-scale model, wherein the portal vein geometric multi-scale model is formed by coupling a three-dimensional model of a portal vein system and a zero-dimensional model of a liver blood circulation system of the patient.

### CROSS-REFERENCE TO RELATED APPLICATION

The present disclosure claims the priority to the Chinese patent application with the filling No. 2024107934877 filed with the Chinese Patent Office on Jun. 19, 2024, and entitled “DETERMINATION METHOD, APPARATUS, ELECTRONIC DEVICE, AND STORAGE MEDIUM FOR POSTOPERATIVE PORTAL VEIN PRESSURE”, the contents of which are incorporated herein by reference in entirety.

### TECHNICAL FIELD

The present disclosure relates to the technical field of medical information data processing, and particularly to a determination method, an apparatus, an electronic device, and a storage medium for a postoperative portal vein pressure.

### BACKGROUND ART

Through the transjugular intrahepatic portosystemic shunt (TIPS), a stent is implanted in a minimally invasive manner into the liver parenchyma between the portal vein and hepatic vein to build a shunt, which structurally and significantly reduces the resistance faced by the portal vein blood flow. This is one of the key measures to reduce portal vein pressure in patients with liver cirrhosis. In clinical practice, the selection for the stent diameter will significantly impact the magnitude of postoperative portal vein pressure and the therapeutic effect: if the selected diameter is too small, it will not effectively reduce the portal vein pressure, thereby failing to achieve the expected outcome of the surgery; if the selected diameter is too large, excessive shunting through the stent will significantly increase the risk of hepatic encephalopathy or even hepatic myelopathy. Therefore, the selection for the TIPS stent diameter is directly related to the efficacy of the interventional surgery and the clinical prognosis of the patient.

Currently, the simulation of the hemodynamic state after TIPS can be achieved by constructing a three-dimensional computational model of the portal vein system. However, this method requires an input of postoperative blood flow velocity as a boundary condition, but there are no technical means to predict the postoperative portal vein pressure after TIPS based solely on preoperative measured data when selecting stents of different diameters. Therefore, how to predict the postoperative portal vein pressure before surgery has become an urgent problem to be solved.

### SUMMARY

A determination method for a postoperative portal vein pressure is provided, wherein the determination method includes: obtaining preoperative imaging data and measurement data of a patient; and inputting the preoperative imaging data and the measurement data of the patient into a portal vein geometric multi-scale model to obtain a postoperative portal vein pressure of the patient output by the portal vein geometric multi-scale model, wherein the portal vein geometric multi-scale model is formed by coupling a three-dimensional model of a portal vein system and a zero-dimensional model of a liver blood circulation system of the patient.

An electronic device is provided, which includes a processor, a memory, and a bus; the memory stores machine-readable instructions that are executed by the processor; the processor communicates with the memory via the bus when the electronic device is in operation, and the machine-readable instructions perform the steps of the determination method for a postoperative portal vein pressure as described above when run by the processor.

A computer-readable storage medium is provided, and a computer program is stored in the computer-readable storage medium, wherein the computer program, when executed by a processor, performs the steps of the above-described determination method for a postoperative portal vein pressure.

### BRIEF DESCRIPTION OF DRAWINGS

To more clearly illustrate the technical solutions of the embodiments of the present disclosure, the following will briefly introduce the drawings used in the embodiments. It should be understood that the following drawings only show some embodiments of the present disclosure, and therefore it should not be regarded as a limitation on the scope. Those ordinary skilled in the art can also obtain other related drawings based on these drawings without inventive effort.

FIG. 1 shows a flowchart of a determination method for a postoperative portal vein pressure provided by an embodiment of the present disclosure;

FIG. 2 shows a flowchart of another determination method for a postoperative portal vein pressure provided by an embodiment of the present disclosure;

FIG. 3 shows a schematic diagram of a portal vein geometric multi-scale model provided by an embodiment of the present disclosure;

FIG. 4 shows a structural schematic diagram of a determination apparatus for a postoperative portal vein pressure provided by an embodiment of the present disclosure; and

FIG. 5 shows a structural schematic diagram of an electronic device provided by an embodiment of the present disclosure.

Reference numerals: 410—determination apparatus; 411—obtaining module; 412—determining module; 500—electronic device; 510—processor; 520—memory; 530—bus.

### DETAILED DESCRIPTION OF EMBODIMENTS

To make the objective, technical solutions, and advantages of the embodiment of the present disclosure clearer, the technical solutions in the embodiment of the present disclosure will be clearly and completely described below in conjunction with the drawings. It should be noted that the drawings in the embodiment of the present disclosure serve the purpose of illustration and description only, and are not intended to limit the scope of protection of the present disclosure. In addition, it should be understood that the schematic drawings are not drawn to a physical scale. The flowcharts used in the present disclosure illustrate operations implemented according to some embodiments of the present disclosure. It should be understood that the operations of the flowchart can be implemented out of sequence, and steps without logical contextual relationships can be reversed in order or implemented simultaneously. Persons skilled in the art, guided by the contents of the present disclosure, may add one or more other operations to the flowchart and may remove one or more operations from the flowchart.

In addition, the described embodiments are only a part of the embodiments of the present disclosure, and not all of them. The components of the embodiments of the present disclosure described and illustrated in the drawings can typically be arranged and designed in various configurations. Therefore, the following detailed description of the embodiments of the present disclosure provided in the drawings is not intended to limit the scope of the present disclosure for which protection is claimed, but merely represents selected embodiments of the present disclosure. Based on the embodiments of the present disclosure, all other embodiments obtained by those skilled in the art without inventive effort shall fall within the protection scope of the present disclosure.

To enable persons skilled in the art to use the contents of the present disclosure in the context of a particular application scenario “for determining postoperative portal vein pressure”, the following embodiments are given. For those skilled in the art, the general principles defined herein can be applied to other embodiments and application scenarios without departing from the spirit and scope of the present disclosure.

The following method, apparatus, electronic device, or computer-readable storage medium of the embodiments of the present disclosure can be applied to any scenario in which the postoperative portal vein pressure needs to be determined. The embodiments of the present disclosure do not limit the specific application scenario, and any solution using a determination method, an apparatus, an electronic device, and a storage medium for a postoperative portal vein pressure provided by the embodiments of the present disclosure is within the scope of protection of the present disclosure.

Based on this, the present disclosure provides a determination method, an apparatus, an electronic device, and a storage medium for a postoperative portal vein pressure. By inputting the preoperative imaging data and measurement data of a patient into a portal vein geometric multi-scale model, which is constructed by coupling a three-dimensional model of the portal vein system and a zero-dimensional model of the liver blood circulation system of the patient, the postoperative portal vein pressure of the patient that is output from the portal vein geometric multi-scale model is obtained. This enables the prediction for postoperative portal vein pressure using preoperative data of the patient. The finally obtained postoperative portal vein pressure can be used to evaluate the surgical efficacy before the actual stent implantation, to provide a reference for selecting the size and model of the stent, and to improve the individualization and precision of subsequent interventional surgical treatment.

The technical solution of the present disclosure is achieved as follows.

In a first aspect, a determination method for a postoperative portal vein pressure is provided in the embodiment of the present disclosure, wherein the determination method includes:
obtaining preoperative imaging data and measurement data of a patient; and
inputting the preoperative imaging data and the measurement data of the patient into a portal vein geometric multi-scale model to obtain a postoperative portal vein pressure of the patient output by the portal vein geometric multi-scale model, wherein the portal vein geometric multi-scale model is formed by coupling a three-dimensional model of a portal vein system and a zero-dimensional model of a liver blood circulation system of the patient.

In one or more embodiments, the step of inputting the preoperative imaging data and the measurement data of the patient into a portal vein geometric multi-scale model to obtain a postoperative portal vein pressure of the patient output by the portal vein geometric multi-scale model includes:
inputting the preoperative imaging data and the measurement data of the patient into a portal vein geometric multi-scale model, performing data processing on the imaging data, and reconstructing the geometric model of the portal vein system of the patient;
meshing the geometric model to obtain a three-dimensional model of the portal vein system of the patient, which can be used for numerical computation in fluid dynamics;
determining a zero-dimensional model of the liver blood circulation system based on the measurement data;
coupling the three-dimensional model with the zero-dimensional model to obtain a boundary condition of the three-dimensional model;
combining the boundary conditions of the three-dimensional model and using a semi-implicit method for a pressure-coupled equation set to solve the Navier-Stokes equations and continuity equations, thereby obtaining flow field parameters of the portal vein system of the patient; and
determining the postoperative portal vein pressure of the patient based on the flow field parameters, and determining the postoperative portal vein pressure of the patient as output data of the portal vein geometric multi-scale model, thereby obtaining a postoperative portal vein pressure of the patient output by the portal vein geometric multi-scale model.

In one or more embodiments, the step of coupling the three-dimensional model with the zero-dimensional model to obtain a boundary condition of the three-dimensional model is realized by:
obtaining flow rate data at an inlet of the three-dimensional model transmitted by the zero-dimensional model;
controlling the three-dimensional model to determine a quotient of the flow rate data and a sectional area at the inlet of the three-dimensional model as an inlet average flow velocity of the three-dimensional model;
obtaining outlet pressure intensity data at an outlet of the three-dimensional model transmitted by the zero-dimensional model; and
using the inlet average flow velocity as an inlet boundary condition and the outlet pressure intensity data as the outlet boundary condition to obtain the boundary conditions of the three-dimensional model.

In one or more embodiments, the method further includes:
determining inlet pressure intensity data based on the boundary conditions;
outputting the inlet pressure intensity data from the inlet of the three-dimensional model and transmitting it to the zero-dimensional model;
controlling the zero-dimensional model to calculate a difference between the pressure intensity data of the three-dimensional model at an upstream point of the inlet and the inlet pressure intensity data to obtain a pressure difference data from the upstream point to the inlet of the three-dimensional model;
determining the quotient of the pressure difference data and a vascular flow resistance of a blood vessel corresponding to the pressure difference data as the flow rate data of the blood vessel; and
continuing to transmit the flow rate data to the inlet of the three-dimensional model to update the inlet boundary conditions of the three-dimensional model.

In one or more embodiments, the method further includes:
controlling the three-dimensional model to obtain the flow rate data at the outlet of the three-dimensional model based on the boundary conditions, and transmitting the flow rate data at the outlet to the zero-dimensional model;
controlling the zero-dimensional model to update the outlet pressure intensity data at the outlet of the three-dimensional model based on the flow rate data at the outlet; and
continuing to transmit the outlet pressure intensity data to the outlet of the three-dimensional model to update the outlet boundary conditions of the three-dimensional model.

In one or more embodiments, the step of determining a zero-dimensional model of the liver blood circulation system based on the measurement data includes:
determining a mean arterial pressure based on a systolic arterial pressure and a diastolic arterial pressure in the measurement data;
determining a blood pressure at a hepatic sinusoid level based on an inferior vena cava pressure and the portal vein pressure in the measurement data;
determining a flow rate of a left portal vein branch and a flow rate of a right portal vein branch based on a flow rate in a middle section of a main branch of the portal vein in the measurement data and a preset distribution ratio;
determining a flow rate of a splenic vein and a flow rate of a superior mesenteric vein based on a diameter of a main portal vein, a diameter of a splenic vein, and a diameter of a superior mesenteric vein in the three-dimensional model; and
obtaining a preset flow rate of a hepatic artery system, and obtaining the zero-dimensional model of the liver blood circulation system based on the mean arterial pressure, the inferior vena cava pressure, the portal vein pressure, the blood pressure at the hepatic sinusoid level, the flow rate of the left portal vein branch, the flow rate of the right portal vein branch, the flow rate of the splenic vein, the flow rate of the superior mesenteric vein, and the preset flow rate of the hepatic artery system.

In one or more embodiments, the step of obtaining the zero-dimensional model of the liver blood circulation system based on the mean arterial pressure, the inferior vena cava pressure, the portal vein pressure, the blood pressure at the hepatic sinusoid level, the flow rate of the left portal vein branch, the flow rate of the right portal vein branch, the flow rate of the splenic vein, the flow rate of the superior mesenteric vein, and the preset flow rate of the hepatic artery system, includes:
determining a difference between the mean arterial pressure and the portal vein pressure as a first parameter, and determining a quotient of the first parameter and the flow rate of the splenic vein as a vascular flow resistance of organs upstream of the portal vein excluding a mesentery;
determining a quotient of the first parameter and the flow rate of the superior mesenteric vein as the vascular flow resistance of the mesentery;
determining a difference between the portal vein pressure and the blood pressure at the hepatic sinusoid level as a second parameter, and determining a quotient of the second parameter and the flow rate of the left portal vein branch as a vascular flow resistance of a perfusion region of the left portal vein branch;
determining a quotient of the second parameter and the flow rate of the right portal vein branch as a vascular flow resistance of a perfusion region of the right portal vein branch;
determining a difference between the mean arterial pressure and the blood pressure at the hepatic sinusoid level as a third parameter, and determining a quotient of the third parameter and the preset flow rate of the hepatic artery system as a vascular flow resistance of the hepatic artery system;
determining a difference between the blood pressure at the hepatic sinusoid level and the inferior vena cava pressure as a fourth parameter;
determining a sum of the preset flow rate of the hepatic artery system, the flow rate of the left portal vein branch, and the flow rate of the right portal vein branch as a fifth parameter;
determining a quotient of the fourth parameter and the fifth parameter as a vascular flow resistance of the hepatic vein system; and
using the vascular flow resistance of organs upstream of the portal vein excluding the mesentery, the vascular flow resistance of the mesentery, the vascular flow resistance of the perfusion region of the left portal vein branch, the vascular flow resistance of the perfusion region of the right portal vein branch, the vascular flow resistance of the hepatic artery system, and the vascular flow resistance of the hepatic vein system as parameters of the zero-dimensional model to obtain the zero-dimensional model of the liver blood circulation system.

In one or more embodiments, the measurement data includes the arterial blood pressure, the inferior vena cava pressure, the portal vein pressure, and the flow velocities or flow rates at the middle section of the main branch of the portal vein.

In one or more embodiments, the step of controlling the three-dimensional model to determine a quotient of the flow rate data and a sectional area at the inlet of the three-dimensional model as an inlet average flow velocity of the three-dimensional model specifically includes:

obtaining a sectional mean flow velocity by dividing the flow rate data by a sectional area at the inlet of the three-dimensional model of the portal vein system, and using the sectional mean flow velocity as the inlet average flow velocity of the three-dimensional model of the portal vein system.

In a second aspect, a determination apparatus for a postoperative portal vein pressure is provided in the embodiment of the present disclosure, wherein the determination apparatus includes:
an obtaining module, configured for obtaining preoperative imaging data and measurement data of a patient; and
a determining module, configured for inputting the preoperative imaging data and the measurement data of the patient into a portal vein geometric multi-scale model to obtain a postoperative portal vein pressure of the patient output by the portal vein geometric multi-scale model, wherein the portal vein geometric multi-scale model is formed by coupling a three-dimensional model of a portal vein system and a zero-dimensional model of a liver blood circulation system of the patient.

In a third aspect, an electronic device is provided in the embodiments of the present disclosure, which includes a processor, a memory, and a bus; the memory stores machine-readable instructions that are executed by the processor; the processor communicates with the memory via the bus when the electronic device is in operation, and the machine-readable instructions perform the steps of the determination method for a postoperative portal vein pressure as described above when run by the processor.

In a fourth aspect, a computer-readable storage medium is provided in the embodiments of the present disclosure, and a computer program is stored in the computer-readable storage medium, wherein the computer program, when executed by a processor, performs the steps of the above-described determination method for a postoperative portal vein pressure.

The embodiments of the present disclosure provide a determination method, an apparatus, an electronic device, and a storage medium for a postoperative portal vein pressure. The determination method includes: obtaining preoperative imaging data and measurement data of a patient; and inputting the preoperative imaging data and the measurement data of the patient into a portal vein geometric multi-scale model to obtain a postoperative portal vein pressure of the patient output by the portal vein geometric multi-scale model, wherein the portal vein geometric multi-scale model is formed by coupling a three-dimensional model of a portal vein system and a zero-dimensional model of a liver blood circulation system of the patient.

In the technical solution of the present disclosure, by inputting the preoperative imaging data and measurement data of a patient into a portal vein geometric multi-scale model, which is constructed by coupling a three-dimensional model of the portal vein system and a zero-dimensional model of the liver blood circulation system of the patient, the postoperative portal vein pressure of the patient is obtained as output from the portal vein geometric multi-scale model. This enables the prediction of postoperative portal vein pressure using preoperative data of the patient. The finally obtained postoperative portal vein pressure can be used to evaluate the surgical efficacy before the actual stent implantation, to provide a reference for selecting the size and model of the stent, and to improve the individualization and precision of subsequent interventional surgical treatment.

To make the above objectives, features, and advantages of the present disclosure more evident and comprehensible, the following preferred embodiments are described in detail with the drawings.

The present disclosure provides a determination method, an apparatus, an electronic device, and a storage medium for a postoperative portal vein pressure. The determination method includes: obtaining preoperative imaging data and measurement data of a patient; and inputting the preoperative imaging data and the measurement data of the patient into a portal vein geometric multi-scale model to obtain a postoperative portal vein pressure of the patient output by the portal vein geometric multi-scale model, wherein the portal vein geometric multi-scale model is formed by coupling a three-dimensional model of a portal vein system and a zero-dimensional model of a liver blood circulation system of the patient.

In the technical solution adopted by the present disclosure, by inputting the preoperative imaging data and measurement data of a patient into a portal vein geometric multi-scale model, which is constructed by coupling a three-dimensional model of the portal vein system and a zero-dimensional model of the liver blood circulation system of the patient, the postoperative portal vein pressure of the patient is obtained as output from the portal vein geometric multi-scale model. This enables the prediction of postoperative portal vein pressure using preoperative data of the patient. The finally obtained postoperative portal vein pressure can be used to evaluate the surgical efficacy before the actual stent implantation, to provide a reference for selecting the size and model of the stent, and to improve the individualization and precision of subsequent interventional surgical treatment.

For a better understanding of the embodiments of the present disclosure, a detailed introduction for a determination method of a postoperative portal vein pressure disclosed in the embodiments of the present disclosure will be presented first.

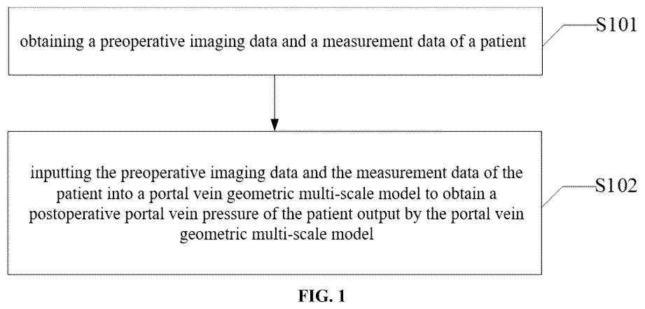

Referring to FIG. 1, FIG. 1 shows a flowchart of a determination method for a postoperative portal vein pressure provided by an embodiment of the present disclosure. As shown in FIG. 1, the determination method includes the following steps.

S101: obtaining preoperative imaging data and measurement data of a patient.

In this step, the obtained preoperative imaging data of the patient is, for example, abdominal enhanced CT venous phase imaging data (DICOM files), and the measurement data of the patient measured before the TIPS stent implantation includes: arterial blood pressure, inferior vena cava pressure, portal vein pressure, and the flow velocity or flow rate at the middle section of the main branch of the portal vein.

S102: inputting the preoperative imaging data and the measurement data of the patient into a portal vein geometric multi-scale model to obtain a postoperative portal vein pressure of the patient output by the portal vein geometric multi-scale model.

In this step, the portal vein geometric multi-scale model is formed by coupling the three-dimensional model of the portal vein system of the patient with the zero-dimensional model of the liver blood circulation system.

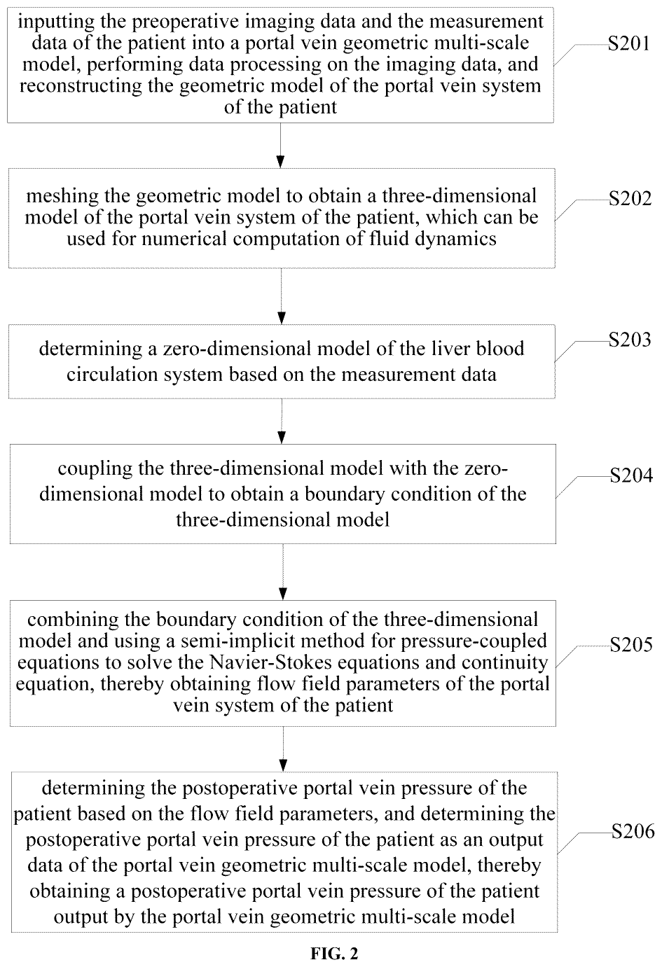

It is important to note that, referring to FIG. 2, where FIG. 2 shows a flowchart of another determination method for a postoperative portal vein pressure provided by an embodiment of the present disclosure, as shown in FIG. 2, the step of inputting the preoperative imaging data and the measurement data of the patient into a portal vein geometric multi-scale model to obtain a postoperative portal vein pressure of the patient output by the portal vein geometric multi-scale model includes the following steps.

S201: inputting the preoperative imaging data and the measurement data of the patient into a portal vein geometric multi-scale model, performing data processing on the imaging data, and reconstructing the geometric model of the portal vein system of the patient.

In this step, the imaging data of the preoperative abdominal enhanced CT venous phase (DICOM files) of the patient are collected as input. Using commercial software such as MIMICS or open-source software such as ITK-SNAP, or other related software, operations such as threshold segmentation, region growing, and smoothing treatment are applied to reconstruct the three-dimensional geometric model of the larger branches of the portal vein system of the patient. Based on this, a geometric model with specified parameters for the TIPS shunt is constructed using commercial software like Geomagic Wrap or other related software.

S202: meshing the geometric model to obtain a three-dimensional model of the portal vein system of the patient, which can be used for numerical computation in fluid dynamics.

In this step, based on the aforementioned geometric model, commercial software such as ANSYS/ICEM or other related software is used to perform computational meshing of the geometric model, to obtain a three-dimensional mesh model used for conducting numerical computation in fluid dynamics. In other words, by obtaining a meshed three-dimensional model of the portal vein system of the patient, the requirement for computational fluid dynamics (CFD) to conduct numerical computation is met.

S203: determining a zero-dimensional model of the liver blood circulation system based on the measurement data.

It is important to note that the step of determining a zero-dimensional model of the liver blood circulation system based on the measurement data includes the following steps.

S2031: determining a mean arterial pressure based on a systolic arterial pressure and a diastolic arterial pressure in the measurement data.

In this step, before TIPS stent placement, the arterial systolic pressure and arterial diastolic pressure of the patient are measured using a monitor, and the mean arterial pressure P_{AO} is calculated.

S2032: determining a blood pressure at a hepatic sinusoid level based on an inferior vena cava pressure and the portal vein pressure in the measurement data.

In this step, the inferior vena cava pressure P_{IVC} and portal vein pressure P_{PV} of the patient are measured using a catheter. The blood pressure at the hepatic sinusoid level P_{SIN} is estimated based on the portal vein pressure P_{PV} and the inferior vena cava pressure P_{IVC}, that is, setting the pressure drops from P_{PV} to P_{SIN} and from P_{SIN} to P_{IVC} in a certain proportion.

S2033: determining a flow rate of a left portal vein branch and a flow rate of a right portal vein branch based on a flow rate in a middle section of a main branch of the portal vein in the measurement data and a preset distribution ratio.

In this step, the flow rate in the middle section of the main branch of the portal vein of the patient is measured using Doppler ultrasound. Based on the diameters of the main branch of the portal vein and its left and right branches in the three-dimensional model of the portal vein system, along with empirical distribution ratios (using Murray's Law, where flow rate at bifurcations is proportional to an exponential power of the diameter e.g. proportional to the third power of the diameter; or using a flow rate distribution ratio averaged across related vascular populations), the flow rate Q_{L} of the left portal vein branch and the flow rate Q_{R} of the right portal vein branch is calculated.

S2034: determining a flow rate of a splenic vein and a flow rate of a superior mesenteric vein based on a diameter of a main portal vein, a diameter of a splenic vein, and a diameter of a superior mesenteric vein in the three-dimensional model.

In this step, similarly, the flow rate Q_{S} of the splenic vein and the flow rate Q_{M} of the superior mesenteric vein are calculated based on the diameters of the main portal vein, the splenic vein, and the superior mesenteric vein in the three-dimensional model of the portal vein system.

S2035: obtaining a preset flow rate of a hepatic artery system, and obtaining the zero-dimensional model of the liver blood circulation system based on the mean arterial pressure, the inferior vena cava pressure, the portal vein pressure, the blood pressure at the hepatic sinusoid level, the flow rate of the left portal vein branch, the flow rate of the right portal vein branch, the flow rate of the splenic vein, the flow rate of the superior mesenteric vein, and the preset flow rate of the hepatic artery system.

In this step, the flow rate of each branch can also be obtained directly by measuring the flow rate of each branch with Doppler ultrasound. Additionally, based on population-averaged data on hepatic artery flow rate for portal hypertension patients (e.g., 9.63 mL/s), the flow rate Q_{HA} in the hepatic artery system (preset flow rate) is set. By summing this flow rate with the total flow rate of the portal vein, the flow rate of the hepatic vein system can be obtained.

It is important to note that, the step of obtaining the zero-dimensional model of the liver blood circulation system based on the mean arterial pressure, the inferior vena cava pressure, the portal vein pressure, the blood pressure at the hepatic sinusoid level, the flow rate of the left portal vein branch, the flow rate of the right portal vein branch, the flow rate of the splenic vein, the flow rate of the superior mesenteric vein, and the preset flow rate of the hepatic artery system, includes the following steps.

1) determining a difference between the mean arterial pressure and the portal vein pressure as a first parameter, and determining a quotient of the first parameter and the flow rate of the splenic vein as a vascular flow resistance of organs upstream of the portal vein excluding a mesentery.

In this step, the calculation formula of the vascular flow resistance of organs upstream of the portal vein excluding a mesentery is as follows:

$${R}_{S} = \frac{{P}_{A O} - {P}_{P V}}{{Q}_{S}} ,$$

where R_{S} is the vascular flow resistance of organs upstream of the portal vein excluding a mesentery, and (P_{AO}-P_{PV}) is the first parameter.

2) determining a quotient of the first parameter and the flow rate of the superior mesenteric vein as the vascular flow resistance of the mesentery.

In this step, the calculation formula of the vascular flow resistance of the mesentery is as follows:

$${R}_{M} = \frac{{P}_{A O} - {P}_{P V}}{{Q}_{M}} ,$$

where R_{M} is the vascular flow resistance of the mesentery.

3) determining a difference between the portal vein pressure and the blood pressure at the hepatic sinusoid level as a second parameter, and determining a quotient of the second parameter and the flow rate of the left portal vein branch as a vascular flow resistance of a perfusion region of the left portal vein branch.

In this step, the calculation formula of the vascular flow resistance of the perfusion region of the left portal vein branch is as follows:

$${R}_{L} = \frac{{P}_{P V} - {P}_{SIN}}{{Q}_{L}} ,$$

where R_{L} is the vascular flow resistance of the perfusion region of the left portal vein branch, and (P_{PV}-P_{SIN}) is the second parameter.

4) determining a quotient of the second parameter and the flow rate of the right portal vein branch as a vascular flow resistance of a perfusion region of the right portal vein branch.

In this step, the calculation formula of the vascular flow resistance of the perfusion region of the right portal vein branch is as follows:

$${R}_{R} = \frac{{P}_{P V} - {P}_{SIN}}{{Q}_{R}} ,$$

where R_{R} is the vascular flow resistance of the perfusion region of the right portal vein branch.

5) determining a difference between the mean arterial pressure and the blood pressure at the hepatic sinusoid level as a third parameter, and determining a quotient of the third parameter and the preset flow rate of the hepatic artery system as a vascular flow resistance of the hepatic artery system.

In this step, the calculation formula of the vascular flow resistance of the hepatic artery system is as follows:

$${R}_{HA} = \frac{{P}_{A O} - {P}_{SIN}}{{Q}_{HA}} ,$$

where R_{HA} is the vascular flow resistance of the hepatic artery system, and (P_{AO}-P_{SIN}) is the third parameter.

6) determining a difference between the blood pressure at the hepatic sinusoid level and the inferior vena cava pressure as a fourth parameter.

In this step, (P_{SIN}-P_{IVC}) is determined as the fourth parameter.

7) determining a sum of the preset flow rate of the hepatic artery system, the flow rate of the left portal vein branch, and the flow rate of the right portal vein branch as a fifth parameter. In this step, (Q_{HA}+Q_{L}+Q_{R}) is determined as the fifth parameter.

8) determining a quotient of the fourth parameter and the fifth parameter as a vascular flow resistance of the hepatic vein system.

In this step, the calculation formula of the vascular flow resistance of the hepatic vein system is as follows:

$${R}_{HV} = \frac{{P}_{SIN} - {P}_{IVC}}{{Q}_{HA} + {Q}_{L} + {Q}_{R}} ;$$

where R_{HV} is the vascular flow resistance of the hepatic vein system.

9) using the vascular flow resistance of organs upstream of the portal vein excluding the mesentery, the vascular flow resistance of the mesentery, the vascular flow resistance of the perfusion region of the left portal vein branch, the vascular flow resistance of the perfusion region of the right portal vein branch, the vascular flow resistance of the hepatic artery system, and the vascular flow resistance of the hepatic vein system as parameters of the zero-dimensional model to obtain the zero-dimensional model of the liver blood circulation system.

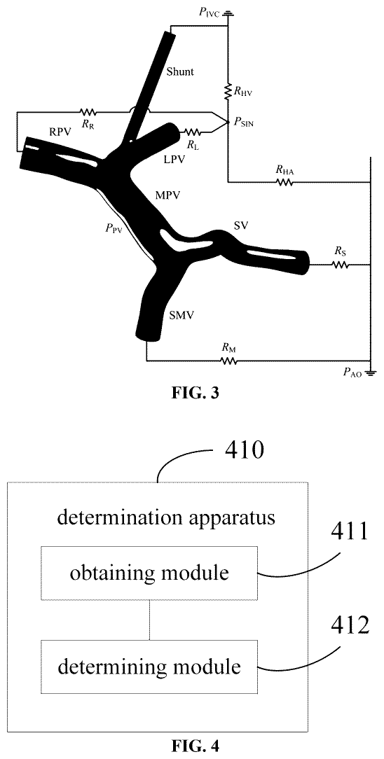

Exemplarily, the portal vein geometric multi-scale model involved in the present embodiment, used for predicting portal vein pressure after TIPS surgery, differs from the three-dimensional computational models used in existing studies (including a computational model of three-dimensional models with zero-dimensional outlet boundary conditions). In the embodiment, a portal vein geometric multi-scale model, constructed by coupling a three-dimensional model of the portal vein system with a zero-dimensional model of the liver blood circulation system, is adopted. The portal vein geometric multi-scale model includes a three-dimensional model of the shunt channel and main vessels of the portal vein system; and a zero-dimensional model of the entire upstream and downstream hepatic circulation. This enables geometric multi-scale simulation of the local small-scale flow field in the TIPS shunt and the large-scale hemodynamic state of the liver blood circulation system. The portal vein geometric multi-scale model can be constructed and parameterized based on preoperative measured clinical data. Based on this, it allows the prediction of postoperative portal vein pressure for shunts of different sizes. The portal vein geometric multi-scale model employs steady-state simulation, with relevant numerical solutions achievable using commercial software such as ANSYS/Fluent, or open-source codes such as OpenFOAM or Sim Vascular. Referring to FIG. 3, a schematic diagram of a portal vein geometric multi-scale model, FIG. 3 is a schematic diagram of a portal vein geometric multi-scale model provided by the embodiment of the present disclosure. As shown in FIG. 3, “SV” represents the splenic vein, “SMV” represents the superior mesenteric vein, “MPV” represents the main branch of the portal vein, “LPV” represents the left portal vein branch, “RPV” represents the right portal vein branch, and “Shunt” represents the shunt. These six names or abbreviations represent corresponding regions within the three-dimensional model of the portal vein system. P_{AO} represents the mean blood pressure of the abdominal aorta, P_{IVC} represents the inferior vena cava pressure, P_{PV} represents the portal vein pressure, P_{SIN} represents the blood pressure at the hepatic sinusoid level, R_{M} represents the vascular flow resistance of the mesentery, R_{S} represents the vascular flow resistance of other organs upstream of the portal vein such as the spleen and stomach, R_{HA} represents the vascular flow resistance of the hepatic artery system, R_{L} represents the vascular flow resistance of the perfusion region of the left portal vein branch, R_{R} represents the vascular flow resistance of the perfusion region of the right portal vein branch, R_{HV} represents the vascular flow resistance of the hepatic vein system, thus forming the portal vein geometric multi-scale model.

S204: coupling the three-dimensional model with the zero-dimensional model to obtain a boundary condition of the three-dimensional model.

It is important to note that, the step of coupling the three-dimensional model with the zero-dimensional model to obtain a boundary condition of the three-dimensional model is realized by the following steps.

S2041: obtaining flow rate data at an inlet of the three-dimensional model transmitted by the zero-dimensional model.

In this step, the flow rate information transferred from the zero-dimensional model (i.e., flow rate passing through R_{S} or R_{M}) is input to the inlet of the three-dimensional model.

S2042: controlling the three-dimensional model to determine a quotient of the flow rate data and a sectional area at the inlet of the three-dimensional model as an inlet average flow velocity of the three-dimensional model.

In this step, by obtaining a sectional mean flow velocity by dividing the flow rate data by a sectional area at the inlet of the three-dimensional model of the portal vein system, and using the sectional mean flow velocity as the inlet average flow velocity of the three-dimensional model of the portal vein system, a paraboloid-shaped flow velocity distribution is set at the inlet to simulate fully developed flow.

S2043: obtaining outlet pressure intensity data at an outlet of the three-dimensional model transmitted by the zero-dimensional model.

In this step, the outlet of the three-dimensional model of the portal vein system inputs the pressure intensity information (outlet pressure intensity data) transmitted by the zero-dimensional model.

S2044: using the inlet average flow velocity as an inlet boundary condition and the outlet pressure intensity data as the outlet boundary condition to obtain the boundary conditions of the three-dimensional model.

It is important to note that the determination method further includes the following steps.

(1) determining inlet pressure intensity data based on the boundary conditions.

(2) outputting the inlet pressure intensity data from the inlet of the three-dimensional model and transmitting it to the zero-dimensional model.

(3) controlling the zero-dimensional model to calculate a difference between the pressure intensity data of the three-dimensional model at an upstream point of the inlet and the inlet pressure intensity data to obtain a pressure difference data from the upstream point to the inlet of the three-dimensional model.

(4) determining the quotient of the pressure difference data and a vascular flow resistance of a blood vessel corresponding to the pressure difference data as the flow rate data of the blood vessel.

(5) continuing to transmit the flow rate data to the inlet of the three-dimensional model to update the inlet boundary conditions of the three-dimensional model.

The above steps (1) to (5) are iterative steps for continuously updating the inlet boundary conditions. The inlet of the three-dimensional model of the portal vein system outputs the inlet pressure intensity data obtained from numerical calculations and transmits it to the zero-dimensional model. The zero-dimensional model calculates the pressure difference based on the inlet pressure intensity data combined with the pressure at its upstream point, and by dividing the pressure difference by the corresponding vascular flow resistance, it obtains the flow rate data of the route (flow rate data of the blood vessel). The flow rate data of the route is then transmitted to the inlet of the three-dimensional model of the portal vein system to update the inlet average flow velocity, thereby updating the inlet boundary conditions.

It is important to note that the determination method further includes the following steps.

[1] controlling the three-dimensional model to obtain the flow rate data at the outlet of the three-dimensional model based on the boundary conditions, and transmitting the flow rate data at the outlet to the zero-dimensional model.

[2] controlling the zero-dimensional model to update the outlet pressure intensity data at the outlet of the three-dimensional model based on the flow rate data at the outlet.

[3] continuing to transmit the outlet pressure intensity data to the outlet of the three-dimensional model to update the outlet boundary conditions of the three-dimensional model.

The above steps [1] to [3] are iterative steps for continuously updating the outlet boundary conditions. The outlet of the three-dimensional model of the portal vein system outputs the flow rate data obtained from numerical calculations and transmits it to the zero-dimensional model. Based on the flow rate data, the zero-dimensional model, combined with each vascular flow resistance and pressure at various points, calculates the outlet pressure intensity data at the outlet of the three-dimensional model of the portal vein system, which is then continuously transmitted to the outlet of the three-dimensional model of the portal vein system to update the outlet boundary conditions.

S205: combining the boundary conditions of the three-dimensional model and using a semi-implicit method for a pressure-coupled equation set to solve the Navier-Stokes equations and continuity equations, thereby obtaining flow field parameters of the portal vein system of the patient.

In this step, the boundary conditions and the computational parameters are set using commercial software ANSYS/Fluent or other relevant software (utilizing the default settings of the steady-state laminar solver in ANSYS/Fluent, where the fluid properties are set to human blood). The control equations, i.e., the three-dimensional steady-state Navier-Stokes equations (N-S equation) and the continuity equations, are numerically solved using the semi-implicit method of a pressure-coupled equation set (SIMPLE algorithm), to output fundamental physical quantities in the entire flow field, such as flow velocity and pressure intensity (flow field parameters).

S206: determining the postoperative portal vein pressure of the patient based on the flow field parameters, and determining the postoperative portal vein pressure of the patient as output data of the portal vein geometric multi-scale model, thereby obtaining a postoperative portal vein pressure of the patient output by the portal vein geometric multi-scale model.

Through the flow field parameters obtained in step S205, the post-TIPS portal vein pressure is determined. The output data of the portal vein geometric multi-scale model includes: the predicted post-TIPS portal vein pressure in a case of the specified stent size and the physical quantities (flow field parameters) in the entire flow field. Utilizing the finally obtained predicting result of the post-TIPS portal vein pressure can enable an assessment of the efficacy of the TIPS procedure before the actual stent is implanted, thus providing reference for selecting size and model of the stent and promoting the individualization and precision of TIPS treatment.

In summary, the data, utilized by the portal vein geometric multi-scale model provided in the embodiment, are pre-TIPS clinical data for model construction and parameter setting. Therefore, the portal vein geometric multi-scale model can achieve construction and computation before actual TIPS stent is implanted. Moreover, in the portal vein geometric multi-scale model, the boundary conditions of the three-dimensional model of the portal vein system are updated based on the calculation results from the zero-dimensional model, rather than specified fixed values. Therefore, the portal vein geometric multi-scale model can simulate the changes in flow rate in the major blood vessels of the portal vein system due to the TIPS procedure, without the need to apply postoperative measured data to the boundary conditions. Accordingly, the method for determining portal vein pressure provided in the embodiment using the portal vein geometric multi-scale model can enable simulation for the postoperative hemodynamic state based on clinically measured pre-TIPS data, and can be used to predict the post-TIPS portal vein pressure under condition of a specified size of the stent shunt.

The embodiment of the present disclosure provides a determination method for a postoperative portal vein pressure. The determination method includes: obtaining preoperative imaging data and measurement data of a patient; and inputting the preoperative imaging data and the measurement data of the patient into a portal vein geometric multi-scale model to obtain a postoperative portal vein pressure of the patient output by the portal vein geometric multi-scale model, wherein the portal vein geometric multi-scale model is formed by coupling a three-dimensional model of a portal vein system and a zero-dimensional model of a liver blood circulation system of the patient.

In the technical solution adopted by the present disclosure, by inputting the preoperative imaging data and measurement data of a patient into a portal vein geometric multi-scale model, which is constructed by coupling a three-dimensional model of the portal vein system and a zero-dimensional model of the liver blood circulation system of the patient, the postoperative portal vein pressure of the patient is obtained as output from the portal vein geometric multi-scale model. This enables the prediction of postoperative portal vein pressure using preoperative data of the patient. The finally obtained postoperative portal vein pressure can be used to evaluate the surgical efficacy before the actual stent implantation, to provide a reference for selecting the size and model of the stent, and to improve the individualization and precision of subsequent interventional surgical treatment.

Based on the same inventive concept, the embodiments of the present disclosure further provide a determination apparatus for a postoperative portal vein pressure corresponding to the above determination method for a postoperative portal vein pressure. Since the principle by which the apparatus of the present disclosure resolves issues is similar to that of the method in the present disclosure, the implementation of the apparatus can be referred to in the method implementation, and redundant details will not be repeated.

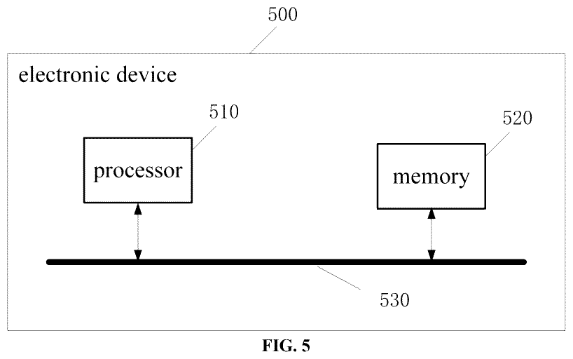

Referring to FIG. 4, FIG. 4 shows a structural schematic diagram of a determination apparatus for a postoperative portal vein pressure provided by an embodiment of the present disclosure. As shown in FIG. 4, the determination apparatus 410 includes:

an obtaining module 411, configured for obtaining preoperative imaging data and measurement data of a patient; and

a determining module 412, configured for inputting the preoperative imaging data and the measurement data of the patient into a portal vein geometric multi-scale model to obtain a postoperative portal vein pressure of the patient output by the portal vein geometric multi-scale model, wherein the portal vein geometric multi-scale model is formed by coupling a three-dimensional model of a portal vein system and a zero-dimensional model of a liver blood circulation system of the patient.

Optionally, when the determining module 412 is configured for inputting the preoperative imaging data and the measurement data of the patient into a portal vein geometric multi-scale model to obtain a postoperative portal vein pressure of the patient output by the portal vein geometric multi-scale model, the determining module 412 is specifically configured for:
inputting the preoperative imaging data and the measurement data of the patient into a portal vein geometric multi-scale model, performing data processing on the imaging data, and reconstructing the geometric model of the portal vein system of the patient;
meshing the geometric model to obtain a three-dimensional model of the portal vein system of the patient, which can be used for numerical computation in fluid dynamics;
determining a zero-dimensional model of the liver blood circulation system based on the measurement data;
coupling the three-dimensional model with the zero-dimensional model to obtain a boundary condition of the three-dimensional model;
combining the boundary conditions of the three-dimensional model and using a semi-implicit method for a pressure-coupled equation set to solve the Navier-Stokes equations and continuity equations, thereby obtaining flow field parameters of the portal vein system of the patient; and
determining the postoperative portal vein pressure of the patient based on the flow field parameters, and determining the postoperative portal vein pressure of the patient as output data of the portal vein geometric multi-scale model, thereby obtaining a postoperative portal vein pressure of the patient output by the portal vein geometric multi-scale model.

Optionally, when the determining module 412 is configured for coupling the three-dimensional model with the zero-dimensional model to obtain a boundary condition of the three-dimensional model, the determining module 412 is specifically configured for:
obtaining flow rate data at an inlet of the three-dimensional model transmitted by the zero-dimensional model;
controlling the three-dimensional model to determine a quotient of the flow rate data and a sectional area at the inlet of the three-dimensional model as an inlet average flow velocity of the three-dimensional model;
obtaining outlet pressure intensity data at an outlet of the three-dimensional model transmitted by the zero-dimensional model; and
using the inlet average flow velocity as an inlet boundary condition and the outlet pressure intensity data as the outlet boundary condition to obtain the boundary conditions of the three-dimensional model.

Optionally, the determining module 412 is further configured for determining inlet pressure intensity data based on the boundary conditions;
outputting the inlet pressure intensity data from the inlet of the three-dimensional model and transmitting it to the zero-dimensional model;
controlling the zero-dimensional model to calculate a difference between the pressure intensity data of the three-dimensional model at an upstream point of the inlet and the inlet pressure intensity data to obtain a pressure difference data from the upstream point to the inlet of the three-dimensional model;
determining the quotient of the pressure difference data and a vascular flow resistance of a blood vessel corresponding to the pressure difference data as the flow rate data of the blood vessel; and
continuing to transmit the flow rate data to the inlet of the three-dimensional model to update the inlet boundary conditions of the three-dimensional model.

Optionally, the determining module 412 is further configured for
controlling the three-dimensional model to obtain the flow rate data at the outlet of the three-dimensional model based on the boundary conditions, and transmitting the flow rate data at the outlet to the zero-dimensional model;
controlling the zero-dimensional model to update the outlet pressure intensity data at the outlet of the three-dimensional model based on the flow rate data at the outlet; and
continuing to transmit the outlet pressure intensity data to the outlet of the three-dimensional model to update the outlet boundary conditions of the three-dimensional model.

Optionally, when the determining module 412 is configured for determining a zero-dimensional model of the liver blood circulation system based on the measurement data, the determining module 412 is specifically configured for:
determining a mean arterial pressure based on a systolic arterial pressure and a diastolic arterial pressure in the measurement data;
determining a blood pressure at a hepatic sinusoid level based on an inferior vena cava pressure and the portal vein pressure in the measurement data;
determining a flow rate of a left portal vein branch and a flow rate of a right portal vein branch based on a flow rate in a middle section of a main branch of the portal vein in the measurement data and a preset distribution ratio;
determining a flow rate of a splenic vein and a flow rate of a superior mesenteric vein based on a diameter of a main portal vein, a diameter of a splenic vein, and a diameter of a superior mesenteric vein in the three-dimensional model; and
obtaining a preset flow rate of a hepatic artery system, and obtaining the zero-dimensional model of the liver blood circulation system based on the mean arterial pressure, the inferior vena cava pressure, the portal vein pressure, the blood pressure at the hepatic sinusoid level, the flow rate of the left portal vein branch, the flow rate of the right portal vein branch, the flow rate of the splenic vein, the flow rate of the superior mesenteric vein, and the preset flow rate of the hepatic artery system.

Optionally, when the determining module 412 is configured for obtaining the zero-dimensional model of the liver blood circulation system based on the mean arterial pressure, the inferior vena cava pressure, the portal vein pressure, the blood pressure at the hepatic sinusoid level, the flow rate of the left portal vein branch, the flow rate of the right portal vein branch, the flow rate of the splenic vein, the flow rate of the superior mesenteric vein, and the preset flow rate of the hepatic artery system, the determining module 412 is specifically configured for:
determining a difference between the mean arterial pressure and the portal vein pressure as a first parameter, and determining a quotient of the first parameter and the flow rate of the splenic vein as a vascular flow resistance of organs upstream of the portal vein excluding a mesentery;
determining a quotient of the first parameter and the flow rate of the superior mesenteric vein as the vascular flow resistance of the mesentery;
determining a difference between the portal vein pressure and the blood pressure at the hepatic sinusoid level as a second parameter, and determining a quotient of the second parameter and the flow rate of the left portal vein branch as a vascular flow resistance of a perfusion region of the left portal vein branch;
determining a quotient of the second parameter and the flow rate of the right portal vein branch as a vascular flow resistance of a perfusion region of the right portal vein branch;
determining a difference between the mean arterial pressure and the blood pressure at the hepatic sinusoid level as a third parameter, and determining a quotient of the third parameter and the preset flow rate of the hepatic artery system as a vascular flow resistance of the hepatic artery system;
determining a difference between the blood pressure at the hepatic sinusoid level and the inferior vena cava pressure as a fourth parameter;
determining a sum of the preset flow rate of the hepatic artery system, the flow rate of the left portal vein branch, and the flow rate of the right portal vein branch as a fifth parameter;
determining a quotient of the fourth parameter and the fifth parameter as a vascular flow resistance of the hepatic vein system; and
using the vascular flow resistance of organs upstream of the portal vein excluding the mesentery, the vascular flow resistance of the mesentery, the vascular flow resistance of the perfusion region of the left portal vein branch, the vascular flow resistance of the perfusion region of the right portal vein branch, the vascular flow resistance of the hepatic artery system, and the vascular flow resistance of the hepatic vein system as parameters of the zero-dimensional model to obtain the zero-dimensional model of the liver blood circulation system.

The embodiment of the present disclosure provides a determination apparatus for a postoperative portal vein pressure, wherein the determination apparatus includes: an obtaining module, configured for obtaining preoperative imaging data and measurement data of a patient; and a determining module, configured for inputting the preoperative imaging data and the measurement data of the patient into a portal vein geometric multi-scale model to obtain a postoperative portal vein pressure of the patient output by the portal vein geometric multi-scale model, wherein the portal vein geometric multi-scale model is formed by coupling a three-dimensional model of a portal vein system and a zero-dimensional model of a liver blood circulation system of the patient.

In the technical solution adopted by the present disclosure, by inputting the preoperative imaging data and measurement data of a patient into a portal vein geometric multi-scale model, which is constructed by coupling a three-dimensional model of the portal vein system and a zero-dimensional model of the liver blood circulation system of the patient, the postoperative portal vein pressure of the patient is obtained as output from the portal vein geometric multi-scale model. This enables the prediction of postoperative portal vein pressure using preoperative data of the patient. The finally obtained postoperative portal vein pressure can be used to evaluate the surgical efficacy before the actual stent implantation, to provide a reference for selecting the size and model of the stent, and to improve the individualization and precision of treatment for subsequent stent placement procedures.

Referring to FIG. 5, FIG. 5 is a structural schematic diagram of an electronic device provided by an embodiment of the present disclosure. As shown in FIG. 5, the electronic device 500 includes a processor 510, a memory 520, and a bus 530, wherein

the memory 520 stores machine-readable instructions executable by the processor 510. When the electronic device 500 operates, the processor 510 communicates with the memory 520 via the bus 530. When the processor 510 executes the machine-readable instructions, it can perform the steps of the determination method for a postoperative portal vein pressure as described in the method embodiments illustrated in FIG. 1 to FIG. 2. Specific implementation details can be found in the method embodiments, which will not be redundantly described here.

The embodiment of the present disclosure further provides a computer-readable storage medium, wherein a computer program is stored on the computer-readable storage medium. When executed by a processor, the computer program can perform the steps of the determination method for a postoperative portal vein pressure as illustrated in the method embodiments in FIG. 1 to FIG. 2. Specific implementation details can be found in the method embodiments, which will not be redundantly described here.

It will be clear to those skilled in the field that, for the convenience and brevity of the description, the specific working processes of the systems, devices, and units described above can be referred to as the corresponding processes in the preceding method embodiments and will not be repeated here.

In the several embodiments provided in the present disclosure, it should be understood that the systems, devices, and methods disclosed can be implemented in other ways. The above-described embodiments of the device are merely schematic, for example, the division of the units described, which is only a logical functional division, can be divided in another way when implemented; also, for example, multiple units or components can be combined or can be integrated into another system, or some features can be ignored, or not implemented. On another point, the mutual coupling, direct coupling, or communication connection, shown or discussed herein, can be an indirect coupling or communication connection through communication interfaces, devices, or units, which can be electrical, mechanical, or other forms.

The units illustrated as separate components can/cannot be physically separated, and the components displayed as units can/cannot be physical units, i.e., they can be located in one place or distributed to a plurality of network units. Some or all of these units can be selected according to actual needs to achieve the objective of the embodiment solution.

In addition, each functional unit in the various embodiments of the present disclosure may be integrated within one processing unit, may physically exist as individual units, or may have two or more units integrated into one.

If the functions are implemented in the form of software functional units and sold or used as standalone products, they can be stored in a non-volatile, computer-readable storage medium executable by a processor. Based on this understanding, the technical solutions of the present disclosure, in essence, or the portions contributing to the prior art or parts of these solutions, can be embodied as a software product. The computer software product is stored in a storage medium and includes several instructions enabling a computing device (such as a personal computer, server, or network device) to execute all or part of the steps of the methods described in the various embodiments of the present disclosure. The aforementioned storage medium includes various media that can store program code, such as USB drives, mobile hard drives, Read-Only Memory (ROM), Random Access Memory (RAM), magnetic disks, or optical disks.

Finally, it should be noted that the embodiments described above are merely specific implementations of the present disclosure, intended to illustrate the technical solutions of the present disclosure and not to limit it. The protection scope of the present disclosure is not confined to these. Although the present disclosure has been described in detail regarding the foregoing embodiments, those skilled in the art should understand that any modifications or easily conceived alterations to the technical solutions described in the embodiments, or equivalent replacements of some technical features, made by those skilled in the art within the disclosed technical scope, do not depart from the spirit and scope of the technical solutions of the embodiments of the present disclosure. All such modifications, alterations, or replacements should be included within the protection scope of the present disclosure. Therefore, the protection scope of the present disclosure shall be of protection of the claims.

### INDUSTRIAL PRACTICALITY

In the technical solution adopted by the present disclosure, by inputting the preoperative imaging data and measurement data of a patient into a portal vein geometric multi-scale model, which is constructed by coupling a three-dimensional model of the portal vein system and a zero-dimensional model of the liver blood circulation system of the patient, the postoperative portal vein pressure of the patient is obtained as output from the portal vein geometric multi-scale model. This enables the prediction for postoperative portal vein pressure using preoperative data of the patient. The finally obtained postoperative portal vein pressure can be used to evaluate the surgical efficacy before the actual stent implantation, to provide a reference for selecting the size and model of the stent, and to improve the individualization and precision of subsequent interventional surgical treatment.

1. A determination method for a postoperative portal vein pressure, wherein the determination method comprises:
obtaining preoperative imaging data and measurement data of a patient; and
inputting the preoperative imaging data and the measurement data of the patient into a portal vein geometric multi-scale model to obtain a postoperative portal vein pressure of the patient output by the portal vein geometric multi-scale model, wherein the portal vein geometric multi-scale model is formed by coupling a three-dimensional model of a portal vein system and a zero-dimensional model of a liver blood circulation system of the patient.

2. The determination method according to claim 1, wherein the step of inputting the preoperative imaging data and the measurement data of the patient into a portal vein geometric multi-scale model to obtain a postoperative portal vein pressure of the patient output by the portal vein geometric multi-scale model comprises:
inputting the preoperative imaging data and the measurement data of the patient into the portal vein geometric multi-scale model, performing data processing on the imaging data, and reconstructing a geometric model of the portal vein system of the patient;
meshing the geometric model to obtain the three-dimensional model of the portal vein system of the patient that can be used for numerical computation in fluid dynamics;
determining the zero-dimensional model of the liver blood circulation system based on the measurement data;
coupling the three-dimensional model with the zero-dimensional model to obtain a boundary condition of the three-dimensional model;
combining the boundary condition of the three-dimensional model and using a semi-implicit method for a pressure-coupled equation set to solve Navier-Stokes equations and continuity equations, thereby obtaining flow field parameters of the portal vein system of the patient; and
determining the postoperative portal vein pressure of the patient based on the flow field parameters, and determining the postoperative portal vein pressure of the patient as output data of the portal vein geometric multi-scale model, thereby obtaining the postoperative portal vein pressure of the patient output by the portal vein geometric multi-scale model.

3. The determination method according to claim 2, wherein the step of coupling the three-dimensional model with the zero-dimensional model to obtain a boundary condition of the three-dimensional model is realized by following steps:
obtaining flow rate data at an inlet of the three-dimensional model transmitted by the zero-dimensional model;
controlling the three-dimensional model to determine a quotient of the flow rate data and a sectional area at the inlet of the three-dimensional model as an inlet average flow velocity of the three-dimensional model;
obtaining outlet pressure intensity data at an outlet of the three-dimensional model transmitted by the zero-dimensional model; and
using the inlet average flow velocity as an inlet boundary condition and the outlet pressure intensity data as the outlet boundary condition to obtain the boundary condition of the three-dimensional model.

4. The determination method according to claim 3, wherein the determination method further comprises:
determining inlet pressure intensity data based on the boundary condition;
outputting the inlet pressure intensity data from the inlet of the three-dimensional model and transmitting it to the zero-dimensional model;
controlling the zero-dimensional model to calculate a difference between pressure intensity data of the three-dimensional model at an upstream point of the inlet and the inlet pressure intensity data to obtain a pressure difference data from the upstream point to the inlet of the three-dimensional model;
determining a quotient of the pressure difference data and a vascular flow resistance of a blood vessel corresponding to the pressure difference data as flow rate data of the blood vessel; and
continuing to transmit the flow rate data to the inlet of the three-dimensional model to update the inlet boundary condition of the three-dimensional model.

5. The determination method according to claim 3, wherein the determination method further comprises:
controlling the three-dimensional model to obtain flow rate data at the outlet of the three-dimensional model based on the boundary condition, and transmitting the flow rate data at the outlet to the zero-dimensional model;
controlling the zero-dimensional model to update the outlet pressure intensity data at the outlet of the three-dimensional model based on the flow rate data at the outlet; and
continuing to transmit the outlet pressure intensity data to the outlet of the three-dimensional model to update the outlet boundary condition of the three-dimensional model.

6. The determination method according to claim 2, wherein the step of determining the zero-dimensional model of the liver blood circulation system based on the measurement data comprises:
determining a mean arterial pressure based on a systolic arterial pressure and a diastolic arterial pressure in the measurement data;
determining a blood pressure at a hepatic sinusoid level based on an inferior vena cava pressure and the portal vein pressure in the measurement data;
determining a flow rate of a left portal vein branch and a flow rate of a right portal vein branch based on a flow rate in a middle section of a main branch of the portal vein in the measurement data and a preset distribution ratio;
determining a flow rate of a splenic vein and a flow rate of a superior mesenteric vein based on a diameter of a main portal vein, a diameter of the splenic vein, and a diameter of the superior mesenteric vein in the three-dimensional model; and
obtaining a preset flow rate of a hepatic artery system, and obtaining the zero-dimensional model of the liver blood circulation system based on the mean arterial pressure, the inferior vena cava pressure, the portal vein pressure, the blood pressure at the hepatic sinusoid level, the flow rate of the left portal vein branch, the flow rate of the right portal vein branch, the flow rate of the splenic vein, the flow rate of the superior mesenteric vein, and the preset flow rate of the hepatic artery system.

7. The determination method according to claim 6, wherein the step of obtaining the zero-dimensional model of the liver blood circulation system based on the mean arterial pressure, the inferior vena cava pressure, the portal vein pressure, the blood pressure at the hepatic sinusoid level, the flow rate of the left portal vein branch, the flow rate of the right portal vein branch, the flow rate of the splenic vein, the flow rate of the superior mesenteric vein, and the preset flow rate of the hepatic artery system, comprises:
determining a difference between the mean arterial pressure and the portal vein pressure as a first parameter, and determining a quotient of the first parameter and the flow rate of the splenic vein as a vascular flow resistance of organs upstream of the portal vein excluding a mesentery;
determining a quotient of the first parameter and the flow rate of the superior mesenteric vein as a vascular flow resistance of the mesentery;
determining a difference between the portal vein pressure and the blood pressure at the hepatic sinusoid level as a second parameter, and determining a quotient of the second parameter and the flow rate of the left portal vein branch as a vascular flow resistance of a perfusion region of the left portal vein branch;
determining a quotient of the second parameter and the flow rate of the right portal vein branch as a vascular flow resistance of a perfusion region of the right portal vein branch;
determining a difference between the mean arterial pressure and the blood pressure at the hepatic sinusoid level as a third parameter, and determining a quotient of the third parameter and the preset flow rate of the hepatic artery system as a vascular flow resistance of the hepatic artery system;
determining a difference between the blood pressure at the hepatic sinusoid level and the inferior vena cava pressure as a fourth parameter;
determining a sum of the preset flow rate of the hepatic artery system, the flow rate of the left portal vein branch, and the flow rate of the right portal vein branch as a fifth parameter;
determining a quotient of the fourth parameter and the fifth parameter as a vascular flow resistance of a hepatic vein system; and
using the vascular flow resistance of organs upstream of the portal vein excluding the mesentery, the vascular flow resistance of the mesentery, the vascular flow resistance of the perfusion region of the left portal vein branch, the vascular flow resistance of the perfusion region of the right portal vein branch, the vascular flow resistance of the hepatic artery system, and the vascular flow resistance of the hepatic vein system as parameters of the zero-dimensional model to obtain the zero-dimensional model of the liver blood circulation system.

8. The determination method according to claim 1, wherein the measurement data comprises an arterial blood pressure, an inferior vena cava pressure, the portal vein pressure, and a flow velocity or a flow rate at a middle section of a main branch of the portal vein.

9. The determination method according to claim 3, wherein the step of controlling the three-dimensional model to determine a quotient of the flow rate data and a sectional area at the inlet of the three-dimensional model as an inlet average flow velocity of the three-dimensional model comprises:
obtaining a sectional mean flow velocity by dividing the flow rate data by the sectional area at the inlet of the three-dimensional model of the portal vein system, and using the sectional mean flow velocity as the inlet average flow velocity of the three-dimensional model of the portal vein system.

10. An electronic device, comprising a processor, a memory, and a bus, wherein the memory stores machine-readable instructions that are executed by the processor, the processor communicates with the memory via the bus when the electronic device is in operation, and the machine-readable instructions perform the steps of the determination method for the postoperative portal vein pressure according to claim 1 when run by the processor.

11. A non-volatile computer-readable storage medium, wherein a computer program is stored on the computer-readable storage medium, and the computer program, when executed by a processor, performs the steps of the determination method for the postoperative portal vein pressure according to claim 1.

12. The electronic device according to claim 10, wherein the step of inputting the preoperative imaging data and the measurement data of the patient into a portal vein geometric multi-scale model to obtain a postoperative portal vein pressure of the patient output by the portal vein geometric multi-scale model comprises:
inputting the preoperative imaging data and the measurement data of the patient into the portal vein geometric multi-scale model, performing data processing on the imaging data, and reconstructing a geometric model of the portal vein system of the patient;
meshing the geometric model to obtain the three-dimensional model of the portal vein system of the patient that can be used for numerical computation in fluid dynamics;
determining the zero-dimensional model of the liver blood circulation system based on the measurement data;
coupling the three-dimensional model with the zero-dimensional model to obtain a boundary condition of the three-dimensional model;
combining the boundary condition of the three-dimensional model and using a semi-implicit method for a pressure-coupled equation set to solve Navier-Stokes equations and continuity equations, thereby obtaining flow field parameters of the portal vein system of the patient; and
determining the postoperative portal vein pressure of the patient based on the flow field parameters, and determining the postoperative portal vein pressure of the patient as output data of the portal vein geometric multi-scale model, thereby obtaining the postoperative portal vein pressure of the patient output by the portal vein geometric multi-scale model.

13. The electronic device according to claim 12, wherein the step of coupling the three-dimensional model with the zero-dimensional model to obtain a boundary condition of the three-dimensional model is realized by following steps:
obtaining flow rate data at an inlet of the three-dimensional model transmitted by the zero-dimensional model;
controlling the three-dimensional model to determine a quotient of the flow rate data and a sectional area at the inlet of the three-dimensional model as an inlet average flow velocity of the three-dimensional model;
obtaining outlet pressure intensity data at an outlet of the three-dimensional model transmitted by the zero-dimensional model; and
using the inlet average flow velocity as an inlet boundary condition and the outlet pressure intensity data as the outlet boundary condition to obtain the boundary condition of the three-dimensional model.

14. The electronic device according to claim 13, wherein the determination method further comprises:
determining inlet pressure intensity data based on the boundary condition;
outputting the inlet pressure intensity data from the inlet of the three-dimensional model and transmitting it to the zero-dimensional model;
controlling the zero-dimensional model to calculate a difference between pressure intensity data of the three-dimensional model at an upstream point of the inlet and the inlet pressure intensity data to obtain a pressure difference data from the upstream point to the inlet of the three-dimensional model;
determining a quotient of the pressure difference data and a vascular flow resistance of a blood vessel corresponding to the pressure difference data as flow rate data of the blood vessel; and
continuing to transmit the flow rate data to the inlet of the three-dimensional model to update the inlet boundary condition of the three-dimensional model.

15. The electronic device according to claim 13, wherein the determination method further comprises:
controlling the three-dimensional model to obtain flow rate data at the outlet of the three-dimensional model based on the boundary condition, and transmitting the flow rate data at the outlet to the zero-dimensional model;
controlling the zero-dimensional model to update the outlet pressure intensity data at the outlet of the three-dimensional model based on the flow rate data at the outlet; and
continuing to transmit the outlet pressure intensity data to the outlet of the three-dimensional model to update the outlet boundary condition of the three-dimensional model.

16. The electronic device according to claim 12, wherein the step of determining the zero-dimensional model of the liver blood circulation system based on the measurement data comprises:
determining a mean arterial pressure based on a systolic arterial pressure and a diastolic arterial pressure in the measurement data;
determining a blood pressure at a hepatic sinusoid level based on an inferior vena cava pressure and the portal vein pressure in the measurement data;
determining a flow rate of a left portal vein branch and a flow rate of a right portal vein branch based on a flow rate in a middle section of a main branch of the portal vein in the measurement data and a preset distribution ratio;
determining a flow rate of a splenic vein and a flow rate of a superior mesenteric vein based on a diameter of a main portal vein, a diameter of the splenic vein, and a diameter of the superior mesenteric vein in the three-dimensional model; and
obtaining a preset flow rate of a hepatic artery system, and obtaining the zero-dimensional model of the liver blood circulation system based on the mean arterial pressure, the inferior vena cava pressure, the portal vein pressure, the blood pressure at the hepatic sinusoid level, the flow rate of the left portal vein branch, the flow rate of the right portal vein branch, the flow rate of the splenic vein, the flow rate of the superior mesenteric vein, and the preset flow rate of the hepatic artery system.

17. The non-volatile computer-readable storage medium according to 11, wherein the step of inputting the preoperative imaging data and the measurement data of the patient into a portal vein geometric multi-scale model to obtain a postoperative portal vein pressure of the patient output by the portal vein geometric multi-scale model comprises:
inputting the preoperative imaging data and the measurement data of the patient into a portal vein geometric multi-scale model, performing data processing on the imaging data, and reconstructing a geometric model of the portal vein system of the patient;
meshing the geometric model to obtain the three-dimensional model of the portal vein system of the patient that can be used for numerical computation in fluid dynamics;
determining the zero-dimensional model of the liver blood circulation system based on the measurement data;
coupling the three-dimensional model with the zero-dimensional model to obtain a boundary condition of the three-dimensional model;
combining the boundary condition of the three-dimensional model and using a semi-implicit method for a pressure-coupled equation set to solve Navier-Stokes equations and continuity equations, thereby obtaining flow field parameters of the portal vein system of the patient; and
determining the postoperative portal vein pressure of the patient based on the flow field parameters, and determining the postoperative portal vein pressure of the patient as output data of the portal vein geometric multi-scale model, thereby obtaining a postoperative portal vein pressure of the patient output by the portal vein geometric multi-scale model.

18. The non-volatile computer-readable storage medium according to 17, wherein the step of coupling the three-dimensional model with the zero-dimensional model to obtain a boundary condition of the three-dimensional model is realized by following steps:
obtaining flow rate data at an inlet of the three-dimensional model transmitted by the zero-dimensional model;
controlling the three-dimensional model to determine a quotient of the flow rate data and a sectional area at the inlet of the three-dimensional model as an inlet average flow velocity of the three-dimensional model;
obtaining outlet pressure intensity data at an outlet of the three-dimensional model transmitted by the zero-dimensional model; and
using the inlet average flow velocity as an inlet boundary condition and the outlet pressure intensity data as an outlet boundary condition to obtain the boundary condition of the three-dimensional model.

19. The non-volatile computer-readable storage medium according to 18, wherein the determination method further comprises:
determining inlet pressure intensity data based on the boundary condition;
outputting the inlet pressure intensity data from the inlet of the three-dimensional model and transmitting it to the zero-dimensional model;
controlling the zero-dimensional model to calculate a difference between pressure intensity data of the three-dimensional model at an upstream point of the inlet and the inlet pressure intensity data to obtain a pressure difference data from the upstream point to the inlet of the three-dimensional model;
determining a quotient of the pressure difference data and a vascular flow resistance of a blood vessel corresponding to the pressure difference data as flow rate data of the blood vessel; and
continuing to transmit the flow rate data to the inlet of the three-dimensional model to update the inlet boundary condition of the three-dimensional model.

20. The non-volatile computer-readable storage medium according to 18, wherein the determination method further comprises:
controlling the three-dimensional model to obtain flow rate data at the outlet of the three-dimensional model based on the boundary condition, and transmitting the flow rate data at the outlet to the zero-dimensional model;
controlling the zero-dimensional model to update the outlet pressure intensity data at the outlet of the three-dimensional model based on the flow rate data at the outlet; and
continuing to transmit the outlet pressure intensity data to the outlet of the three-dimensional model to update the outlet boundary condition of the three-dimensional model.
